(19) United States
(12) Patent Application Publication
(10) Pub. No.: US 2010/0280358 A1
(43) Pub. Date: Nov. 4, 2010

(54) SINGLE BREATH-HOLD SYSTEM AND METHOD FOR DETECTION AND ASSESSMENT OF MULTI-ORGAN PHYSIOLOGIC, MORPHOLOGIC AND STRUCTURAL CHANGES

(75) Inventors: Jaime F. MATA, Crozet, VA (US); Kai Ruppert, Keswick, VA (US); John P. Mugler, Charlottsville, VA (US)

Correspondence Address: BLANK ROME LLP, WATERGATE, 600 NEW HAMPSHIRE AVENUE, N.W., WASHINGTON, DC, 20037, US

(73) Assignee: University of Virginia Patent Foundation, Charlottesville, VA (US)

(21) Appl. No.: 12/771,767

(22) Filed: Apr. 30, 2010

Related U.S. Application Data
(60) Provisional application No. 61/174,657, filed on May 1, 2009.

(51) Int. Cl. A61B 5/055; G06T 7/00

(52) U.S. Cl. 600/420; 382/131

(57) ABSTRACT

In a single breath-hold the physiologic, morphologic and structural changes in multiple organs are detected and assessed. The main steps process include: polarizing the Xe-129 gas; inhalation or introduction of a certain pre-calculated amount of the gas and start of the breath-hold; acquisition of multiple spatially oriented spectrums, localized in the same plane (2D), in multiple different planes (3D) or in 3D plus at multiple time intervals (4D); stop the breath-hold; post-processing of the acquired data; evaluation of the multiple spectrums by comparison of the values in a region of interest with those in surrounding tissues or known as normal.

### REFERENCE TO RELATED APPLICATION

The present application claims the benefit of U.S. Provisional Patent Application No. 61/174,657, filed May 1, 2009, whose disclosure is hereby incorporated by reference in its entirety into the present disclosure.

### STATEMENT OF GOVERNMENT INTEREST

This invention was made with government support under Grant Nos. R01 EB003202 and R01 HL079077 awarded by National Institutes of Health. The government has certain rights in the invention.

### DESCRIPTION OF RELATED ART

Hyperpolarized xenon-129 (hp Xe-129) and hyperpolarized helium-3 (hp He-3) have been used as gaseous magnetic resonance (MR) contrast agents for more than a decade (1-4). Their proven safety (5) and the non-involvement of ionizing radiation make these agents very attractive for medical imaging. Hp He-3 has been the preferred agent for a large number of research centers around the world, due to its easily achievable higher polarizations (6), easy transport across continents even after being polarized and relatively large gyromagnetic ratio (−32.4 MHz/T). Recent higher demand for this gas for medical imaging and for homeland security has driven the prices to three fold increases, and the situation only has a tendency to worsen in the near future, since supply is limited and a global reduction of the number of nuclear war heads is likely, which will shrink the production of He-3 and its supply even further.

In this scenario the use of relatively cheaper isotopically enriched Xe-129 (enrichments >80%) allied with recent increases of the Xe-129 polarization at high output volumes (7,8), plus the intrinsic high solubility characteristics of this gas in water, blood and pulmonary parenchyma (9,10), makes it a very appealing candidate to replace He-3 in MR medical imaging. He-3 has a very low solubility and cannot be used as a probe to study alveolar-parenchyma-blood gas transfer, as can be done with hp Xe-129. Besides the high solubility of the Xe-129 in the blood and lung parenchyma, another attractive property of this gas is its large chemical shift (˜200 ppm) (FIG. 1) between the alveolar gas and the frequencies of the Xe-129 dissolved into the lung parenchyma (tissue ˜197 ppm) and red blood cells (RBC; ˜211 ppm) (FIG. 1), commonly named dissolved-phase (10-14). More specifically, FIG. 1 shows an MR spectrum acquired from a single voxel within a rabbit lung and shows the peak for hp Xe-129 dissolved in red blood cells (RBC) at the center/left; the peak for hp Xe-129 dissolved in tissue at the center/right and the main gas peak at 200 ppm to the far right.

Besides spectroscopic measurements done to characterize the Xe-129 gas uptake and gas exchange process in the lungs of different animal species and in different situations (10-14), three different MR techniques have up-to-now been adapted for hp Xe-129 to image pulmonary gas dynamics, by measuring the amounts of Xe-129 in each of the three distinct resonance frequencies. Driehuys et al. have used an elegant technique based on the Dixon method (13) commonly used to separate tissue from water frequencies. This method also referred by XACT (Xenon Alveolar Capillary Transfer) imaging, can produce images of the dissolved-phase with resolutions around 1×1 mm², as it had been shown in a rat model of pulmonary fibrosis (13). The biggest drawback of this technique is perhaps the requirement of large numbers of breath-holds for the multiple averaging required due to the low signal in the dissolved-phase compartments. Such a technique worked reasonably well in an animal model, where the animal was intubated and connected to a ventilator. But it will be challenging to apply it to a clinical setting, where a single short breath-hold is required. Ruppert et al. adapted a different MR technique for probing the hp Xe-129 gas-exchange process in the lung (10,12). In this method, also called XTC (Xenon Transfer Contrast) the measurements of the gas in the different dissolved-phase compartments is done indirectly by measuring the attenuation of the hp Xe-129 signal in the alveoli (gas in the airspace).

This method has been proven efficient in measuring lung tissue thickening (12), but due to the indirect measurements cannot probe the dissolve-phase compartments in areas of the lung that are not ventilated. At last, Swanson et al. have directly imaged the dissolved-phase compartments using CSI (Chemical Shift Imaging) (14), but due to the large voxel size (poor resolution), lower gas polarization (˜5%) and use of natural abundance xenon (Xe-129=26%), the maps were just a rough representation of the lungs. Also this previous implementation of CSI of the lung with hp Xe-129 required the use of mechanical ventilation, imaging for 8 minutes, multiple breath-holds and averaging, which is not practical for application in humans.

The following patents, applications and publications as listed below and throughout this document are hereby incorporated by reference in their entirety herein.

The devices, systems, compositions, computer program products and methods of various embodiments of the invention disclosed herein may utilize aspects disclosed in the following references, applications, publications and patents and which are hereby incorporated by reference herein in their entirety:

1. Middleton H, Black R D, Saam B, Cates G D, Cofer G P, Guenther R, Happer W, Hedlund L W, Johnson G A, Juvan K, et al. MR imaging with hyperpolarized 3He gas. Magn Reson Med 1995; 33(2):271-275.

2. Albert M S, Cates G D, Driehuys B, Happer W, Saam B, Springer C S, Wishnia A. Biological Magnetic-Resonance-Imaging Using Laser Polarized Xe-129. Nature 1994; 370(6486):199-201.

3. Black R D, Middleton H L, Cates G D, Cofer G P, Driehuys B, Happer W, Hedlund L W, Johnson G A, Shattuck M D, Swartz J C. In vivo He-3 MR images of guinea pig lungs. Radiology 1996; 199(3):867-870.

4. DeLange E E, Brookeman J R, Mugler J P, Daniel T M, Driehuys B, Cates G D. MR imaging of the lungs with hyperpolarized Xe-129 gas. Radiology 1996; 201:405-405.

5. DeLange E, Altes T, Harding D, Harrell F, Mata J, Wright C. Hyperpolarized Gas MR imaging of the lung: Safety assessment of inhaled helium-3. 2003; Chicago, Ill.

6. Kauczor H, Surkau R, Roberts T. MRI using hyperpolarized noble gases. Eur Radiol 1998; 8(5):820-827.

7. Hersman F W, Ruset I C, Ketel S, Muradian I, Covrig S D, Distelbrink J, Porter W, Watt D, Ketel J, Brackett J, Hope A, Patz S. Large production system for hyperpolarized 129Xe for human lung imaging studies. Acad Radiol 2008; 15(6):683-692.

8. Ruset I C, Ketel S, Hersman F W. Optical pumping system design for large production of hyperpolarized. Phys Rev Lett 2006; 96(5):053002.

9. Kilian W, Seifert F, Rinneberg H. Dynamic NMR spectroscopy of hyperpolarized (129)Xe in human brain analyzed by an uptake model. Magn Reson Med 2004; 51(4):843-847.

10. Ruppert K, Mata J F, Wang H T, Tobias W A, Cates G D, Brookeman J R, Hagspiel K D, Mugler J P, 3rd. XTC MRI: sensitivity improvement through parameter optimization. Magn Reson Med 2007; 57(6):1099-1109.

11. Ruppert K, Brookeman J R, Hagspiel K D, Driehuys B, Mugler J P. NMR of hyperpolarized Xe-129 in the canine chest: spectral dynamics during a breath-hold. Nmr in Biomedicine 2000; 13(4):220-228.

12. Ruppert K, Brookeman J R, Hagspiel K D, Mugler J P, 3rd. Probing lung physiology with xenon polarization transfer contrast (XTC). Magn Reson Med 2000; 44(3):349-357.

13. Driehuys B, Cofer G P, Pollaro J, Mackel J B, Hedlund L W, Johnson G A. Imaging alveolar-capillary gas transfer using hyperpolarized 129Xe MRI. Proc Natl Acad Sci USA 2006; 103(48):18278-18283.

14. Swanson S D, Rosen M S, Coulter K P, Welsh R C, Chupp T E. Distribution and dynamics of laser-polarized (129)Xe magnetization in vivo. Magn Reson Med 1999; 42(6):1137-1145.

15. Cates G D, Barton A, Bogorad P, Driehuys B, Gatzke M, Happer W, Newbury NR, Saam B. Magnetic-Resonance Experiments with Laser Polarized Rare-Gases. Abstracts of Papers of the American Chemical Society 1991; 202:41-Phys.

16. Driehuys B, Cates G D, Happer W, Mabuchi H, Saam B, Albert M S, Wishnia A. Spin Transfer between Laser-Polarized Xe-129 Nuclei and Surface Protons. Physics Letters A 1993; 184(1):88-92.

17. U.S. Pat. No. 6,808,699 to Driehuys et al., Oct. 26, 2004, “Methods for Imaging Pulmonary and Cardiac Vasculature and Evaluating Blood Flow Using Dissolved Polarized 129XE”.

18. U.S. Pat. No. 6,696,040 to Driehuys, Feb. 24, 2004, “Diagnostic Procedures Using 129XE Spectroscopy Characteristic Chemical Shift to Detect Pathology In Vivo.

19. U.S. Pat. No. 6,991,777 to Driehuys et al., Jan. 31, 2006, “Methods for In Vivo Evaluation of Physiological Conditions and/or Organ or System Function Including Methods to Evaluate Cardiopulmonary Disorders Such as Chronic Heart Failure Using Polarized 129 XE.

20. U.S. Pat. No. 6,593,144 to Albert et al., Jul. 15, 2003, “Magnetic Resonance Imaging Using Hyperpolarized Noble Gases”.

21. U.S. Pat. No. 5,245,282 to Mugler, III, et al., Sep. 14, 1993, “Three-Dimensional Magnetic Resonance Imaging”.

22. U.S. Pat. No. 6,775,568 to Mugler, III, et al., Aug. 10, 2004, “Exchange-Based NMR Imaging and Spectroscopy of Hyperpolarized Xenon-129”.

23. U.S. Pat. No. 7,164,268 to Mugler, III, et al., Jan. 16, 2007, “Method and Apparatus for Spin-Echo-Train MR Imaging Using Prescribed Signal Evolutions.

24. U.S. patent application Ser. No. 12/354,471, Mugler, III, et al., Jan. 15, 2009, “Method and Apparatus for Spin-Echo-Train MR Imaging Using Prescribed Signal Evolutions.

25. U.S. Pat. No. 7,174,200, to Mugler, III, et al., Feb. 6, 2007, “Optimized High-Speec Magnetic Resonance Imaging Method and System Using Hyperpolarized Noble Gases”.

26. U.S. Pat. No. 7,034,533 to Mugler, III, et al., Apr. 25, 2006, “Method and System for Rapid Magnetic Resonance Imaging of Gases with Reduced Diffusion-Induced Signal Loss”.

27. U.S. Patent Application Publication No. US2007/0225592 to Ruppert, et al., Sep. 27, 2007, “Exchange-Weighted Xenon-129 Nuclear Magnetic Resonance System and Related Method” (U.S. Ser. No. 10/592,200).

28. U.S. Patent Application Publication No. US2007/0197903 to Mugler, III, et al., Aug. 23, 2007, “System and Method for Improved Detection and Assessment of Changes in Lung-Tissue Structure” (U.S. Ser. No. 10/592,186).

29. U.S. patent application Ser. No. 12/144,349, Wang, et al., Jun. 23, 2008, “Method and System for NMR Measurement of the Diffusion of Hyperpolarized Noble Gases in Restricting Media over a Range of Time Scales Using Stimulated Echoes”.

30. U.S. patent application Ser. No. 12/194,919, Mugler, III, Aug. 20, 2008, “Magnetic Resonance Method and Apparatus for Generating Different Weighted Images from the Same Magnetic Resonance Echo Signal Evolution”.

31. U.S. Pat. No. 6,630,126 to Driehuys, et al., Oct. 7, 2003, “Diagnostic Procedures Using Direct Injection of Gaseous Hyperpolarized 129 XE and Associated System and Products”.

32. Mata J, Sheng K, Read P, Ruppert K, Hagspiel K, Altes T, Tobias W, Cates G, Brookeman J, Mugler J III. Chemical shift imaging of the lungs with hyperpolarized Xe-129: Results from a rabbit model of stereotactic lung radiosurgery with and without a radioprotector. Proc. 17th International Society of Magnetic Resonance in Medicine (I.S.M.R.M.), Honolulu, April 2009.

33. Stoyanova R, Brown T R. NMR spectral quantitation by principal component analysis. III. A generalized procedure for determination of lineshape variations. J Magn Reson 2002; 154:163-175.

### SUMMARY OF THE INVENTION

It is therefore an object of the invention to overcome the above-noted deficiencies of known procedures.

It is another object of the invention to do so while permitting imaging within a single breath hold.

To achieve the above and other objects, an aspect of various embodiments may comprise a system, computer program product and method to detect and assess in a single breath-hold the physiologic, morphologic and structural changes in multiple organs. Some of the main steps of this process may include: polarizing the Xe-129 gas; inhalation or introduction of a certain pre-calculated amount of the gas and start of the breath-hold; acquisition of multiple spatially oriented spectrums, localized in the same plane (2D), in multiple different planes (3D) or in 3D plus at multiple time intervals (4D); stop the breath-hold; post-processing of the acquired data; evaluation of the multiple spectrums by comparison of the values in a region of interest with those in surrounding tissues or known as normal. The novel method described here as Single breath-hold 2D, 3D or 4D Chemical Shift Imaging (SB-CSI) with hyperpolarized Xe-129 (hp Xe-129) will likely show very high sensitivity for the detection of a large number of pathologies, with minimal or no side-effects, in a very comfortable and fast way.

This system, computer program product and method may provide improved sensitivity for the detection and monitoring of diseases and conditions, including, but not limited to, tissue inflammation, tumors (malign and benign), other forms of cancer that don't comprise masses, pulmonary embolism and tissue edema. Since hp Xe-129 dissolves into tissue and binds to red blood cells, which carry it throughout the body, it can be used not only to assess changes in the lung but in any other organ. Also since it does not involve the use of ionizing radiation and does not have severe side-effects, it can be used multiple times daily, or repeated as desired to determine and monitor the progression of disease or to evaluate the efficacy of therapy.

### BRIEF DESCRIPTION OF THE DRAWINGS

Preferred embodiments of the invention will be set forth in detail with reference to the drawings, in which:

FIG. 1 is a graph showing an MR spectrum acquired from a single voxel within a rabbit lung;

FIGS. 2A-2J are images from rabbit models of stereotactic radiation therapy with and without a radioprotector;

FIGS. 3A-3J are images from a pulmonary embolism model;

FIG. 4 is a plot of a pulse sequence used in the preferred embodiments;

FIG. 5 is a plot of the tissue/gas and RBC/gas ratios;

FIG. 6A is a plot of a full spectrum, from a single voxel, obtained during a 2D-CSI acquisition;

FIG. 6B shows 2D-CSI maps for each of the peaks indicated in FIG. 6A;

FIG. 6C shows gas-peak (ventilation) images;

FIG. 6D shows tissue peak images;

FIG. 6E shows images from the RBC peak; and

FIG. 7 is a schematic diagram of a system on which the preferred embodiments can be implemented.

### DETAILED DESCRIPTION OF THE PREFERRED EMBODIMENTS

An aspect of various embodiments may comprise of a system, computer program product, and method that involves a new and improved process for detecting and quantifying physiological, morphological and structural changes of one or multiple organs in a single breath-hold by measuring the amounts of hp Xe-129 in different compartments using a combination of magnetic resonance imaging (MRI) and magnetic resonance spectroscopy (MRS). The method (SB-CSI) can be described by the following steps, but not limited thereto:

1. Polarization of the Xe-129 gas: This embodiment requires the use of hyperpolarized Xe-129 (hp Xe-129), wherein we define the term “hyperpolarized” state as a much higher than thermal equilibrium polarization state when the gas is in the static magnetic field used to acquire the spectrums and maps described in step 3 below. Any method to create non-equilibrium nuclear polarization (hyperpolarization) of the Xe-129, including but not limited to, optical pumping spin exchange (1, 2, 15, 16) can be used in this system and method.

2. Inhalation or introduction by other means, including but not limited to an endotracheal tube, of a certain pre-calculated amount of hp Xe-129 gas, immediately followed by a breath-hold: After the subject or other medium to be probed is positioned in an appropriate radio-frequency (RF) coil and within the MRI scanner, the hp Xe-129 gas is inhaled from a plastic bag or introduced by other means into the lung or into the medium to be probed, including but not limited to excised organs, cell cultures or animal carcasses. Inhalation or introduction of the gas also includes, but is not limited to, computer controlled gas systems, manually operated syringes or other manually or computer controlled device.

3. Acquisition of multiple spatially oriented spectrums localized in the same plane (2D), in multiple different planes (3D) or in 3D plus at multiple time intervals (4D) using a Single Breath-hold 2D, 3D or 4D Chemical Shift Imaging (SB-CSI) pulse sequence. This novel SB-CSI pulse sequence is described in detail below. Positioning of the multiple spectrums and respective orientation and localization shall be done before the beginning of the acquisition, and mandates which organ(s) or medium(s) will be probed.

4. Stop the breath-hold: At the end of the data acquisition (step 3) the subject can breathe spontaneously. If more data acquisitions are necessary, steps 2-4 can be repeated as desired.

5. Post-processing of the data acquired in step 3: MR spectrums and chemical shift maps can be produced by using any appropriate mathematical process; this includes, but is not limited to: i) filtering the free-induction decay (FID) or echo data obtained from each voxel; ii) zero fill the data to a higher number of points; iii) Fourier transformation; iv) correction for frequency shifts. Once the MR spectrums for each independent voxel are obtained, the final maps for each hp Xe-129 peak are obtained by, but not limited to, fitting a Lorentzian or Gaussian wave form to each peak followed by an integration of each peak it and/or a combination of peaks fits, as well as but not limited to, a combination of their ratios, differences, sums and multiplications. Maps based on the amplitude, or other peak characteristics, besides the area (integral) of the peak(s) may also be obtained and displayed.

6. Evaluation of the multiple spectrums and/or peak maps: By using any appropriate quantitative or statistical comparisons, for example but not limited to, means and standard deviations, values in a region of interest can be compared with those in surrounding tissues or known as normal. These values can be used to state the physiologic, morphologic and structural characteristics of the region of interest and of other tissues being probed by the SB-CSI method. For applications such as, but not limited to, the monitoring of disease progression and/or evaluation of therapy efficacy, all the steps 1-6 can be repeated as desired to determine temporal variations in the regions of interest.

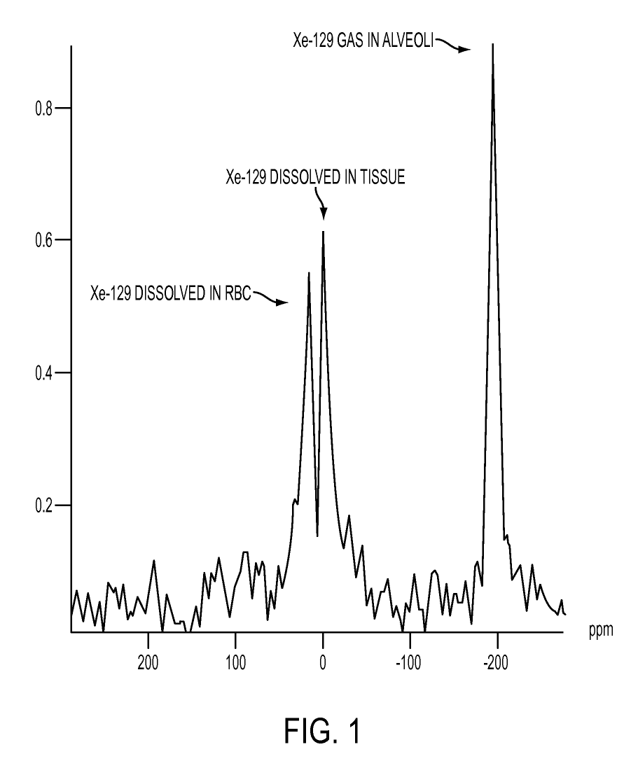

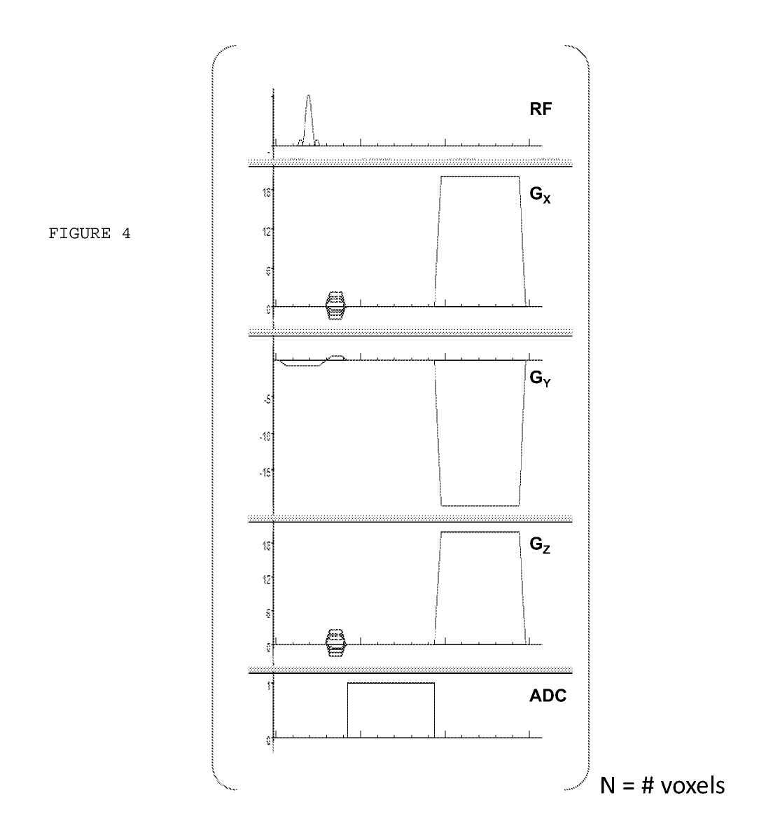

The Single Breath-hold 2D, 3D or 4D Chemical Shift Imaging (SB-CSI), which uses hyperpolarized Xe-129 (hp Xe-129) as a MR probing agent, uses a MR pulse sequence capable of producing complex high-resolution (in-plane voxel size smaller than 3.0×3.0 mm²) CSI maps in multiple orientations, multiple planes and at multiple acquisition times, in a single breath-hold (less than 15 seconds). An example of such a pulse sequence is shown in FIG. 4. This method exploits the fact that, once inhaled, hp Xe-129 dissolves in the lung tissue (parenchyma) and also diffuses and binds to the red blood cells that bathe the lung parenchyma. Once dissolved, hp Xe-129 produces distinct MR signals (“peaks”) that are shifted in resonant frequency from the main gaseous hp Xe-129 peak (FIG. 1). For example, the resonant frequencies of hp Xe-129 dissolved in the lung parenchyma and in the blood differ by 197 ppm and 202 ppm, respectively at 1.5 Tesla, from the frequency of the gas peak (FIG. 1). Also since the hp Xe-129 is carried away in the blood, it can be used to probe any organ in the body. Thus, the physical location of the hp Xe-129, and the relative amount in each compartment (in blood, different tissues, alveolar airspaces, etc) can be deduced from the frequency and other metrics of its signal. These features can provide valuable information regarding regional physiology, morphology and structure of any organ.

In order to create a single breath-hold CSI pulse sequence with high-resolution and large number of voxels (more than 16×16 in-plane matrix voxels), we modified and optimized a standard CSI pulse sequence for use with, but not limited to, hp Xe-129. Since the longitudinal magnetization of the hp Xe-129 spins do not recover after being tipped by a RF pulse, the repetition time (TR) of the pulse sequence can be optimized and reduced to a minimum, which permits much shorter acquisition times for the same number of acquired voxels. A further compaction can be achieved by reducing the time of the dephasing (“crushing”) gradients at the end of each repetition (TR), and by increasing the amplitude of the same gradients, resulting in an identical net area of these, and so keeping the initial effect produced by these gradients. Furthermore, to achieve an acquisition time in the same order as a single breath-hold (less than 15 seconds) and optimize the use of the available hp Xe-129 signal, we applied other novel approaches like, but not limited to, a reduction of the echo time (TE) to less than 2.1 milliseconds, and the use of a small RF flip angle (FA) of less than 30 degrees, per voxel for the tipping magnetization, instead of the traditional large FA's of more than 30 degrees (17) used in this type of spectroscopic measurements. This SB-CSI pulse sequence can be used with a Cartesian K-space acquisition or with non-Cartesian K-space acquisitions, like but not limited to spiral, radial or weighted k-space acquisitions. The use of non-Cartesian K-space acquisitions can be used to increase even further the spatial and/or temporal resolutions. The duration and profile of the FA can also be customized to excite just the hp Xe-129 peaks of interest at a certain magnetic field.

After such modifications the result is a high-resolution CSI pulse sequence tailored for hp Xe-129, and with acquisition times that are now perfectly manageable for a single breath-hold even for small animals like rats and perfectly acceptable in humans even with advanced pulmonary disease, including but not limited to, Chronic Obstructive Pulmonary disease (COPD), emphysema, pulmonary embolism (PE), lung cancer, cystic fibrosis (CF), asthma and sickle cell disease.

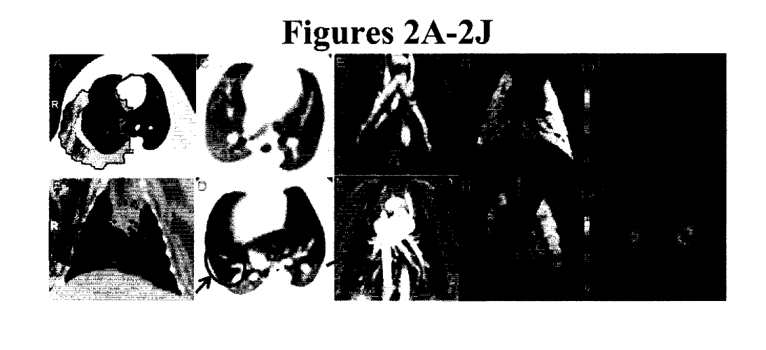

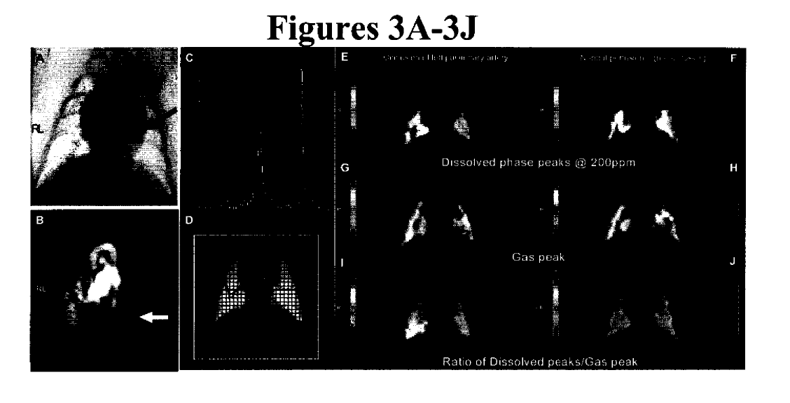

Furthermore, we report the preliminary evaluation of this SB-CSI method using two rabbit models, one of stereotatic radiation therapy with and without a radioprotector (FIGS. 2A-2J), the other a pulmonary embolism model (FIGS. 3A-3J). From the SB-CSI data we directly calculated images reflecting, but not limited to, the amount of hp Xe-129 in the airspaces, and dissolved in the lung tissue and blood, and thus obtained detailed spatial information regarding how hp-Xe-129 is distributed in the different compartments, providing regional information about organ physiology, morphology and structure.

In order to study the efficacy of this technique for detecting changes in lung physiology and morphology, it was applied to a rabbit model of stereotactic lung radiosurgery with and without a radioprotector. The lower lobes of the right lung of nine New Zealand rabbits were irradiated with 3 doses of 11 Gy each (FIGS. 2A, 2B) using a TomoTherapy Hi-Art scanner. The healthy left lung was spared from radiation, so it could be used as a control and also would improve the animal's quality of life. Six of these nine animals received 50 mg/Kg of the radioprotector Amifostine, through an I.V., 20 minutes before each radiation dose. SB-CSI was performed using a 1.5-Tesla clinical MR scanner at baseline, and at 4 and 8 weeks post-radiation treatment. For correlation with the SB-CSI results, each animal was also scanned at the same time points (in the same MR scanner) using a 3D contrast-enhanced MRA sequence (ce-MRA), with 3 cc of a gadolinium chelate (FIGS. 2E, 2F). Within a week of the Xe-129 scan, a hyperpolarized Helium-3 (hp He-3) ventilation scan (FIGS. 2G, 2H) was obtained using the same inhalation and breath-hold technique as those for Xe-129, and a computed tomography (CT) scan was also obtained with a resolution of 0.5×0.5×1.5 mm³ (FIGS. 2C, 2D).

SB-CSI maps based on the integrals of each individual peak were calculated separately for each animal and for each time point. At baseline the mean differences between the lungs were statistically identical for both groups, with 1.3%±1.0% (mean±SD) for the control group (radiosurgery only) and 3.2%±3.5% for the group that received radiosurgery and the radioprotector. At 4 weeks post radiosurgery, the mean differences between the lungs were 6.2%±2.3% and 4.6%±3.2% for the control and radioprotector groups, respectively. At 8 weeks post, the mean difference between the lungs increased to 16.7%±3.2% for the control group, while for the animals that received the radioprotector the value remained identical to baseline, at 3.4%±2.6%. These SB-CSI results of the pulmonary tissue changes correlate well with the focal perfusion and tissue-density changes observed with multiple modalities including ce-MRA and CT scans (FIGS. 2D, 2F). He-3 images showed normal ventilation throughout the lungs for all animals. The results from these multiple modalities lead to the conclusion that ventilation-perfusion mismatches developed in the irradiated areas, which is well supported and characterized by the SB-CSI technique showing a focal increase of the hp Xe-129 dissolved in the lung tissue (FIG. 2J, arrow). SB-CSI detected radiation-induced pulmonary injury at 4 and 8 weeks post treatment, and was able to detect reduced early treatment toxicity as a result of a clinically used radioprotector.

More specifically, in FIGS. 2A-2J, the top row (FIGS. 2A, C, E, G, I) relates to the animal from the radiation plus radioprotector group, while the bottom row (FIGS. 2B, D, F, H, J) relates to the animal from the control group (radiation only). A & B: CT scans with radiation treatment planning superimposed. Area in red received full radiation dose (3×11Gy). All animals received radiation in the same location. C & D: CT scans at 8 weeks post radiation. While lung in C has homogeneous normal tissue density, the lung in D shows an area of hypo-density that correlates with the irradiated area (oval and arrow). E & F: ce-MRA at 8 weeks post radiation. At the same location as in the CT scan (D), the ce-MRA (F) shows evidence of low pulmonary perfusion (arrow). G & H: He-3 ventilation images at 8 weeks post radiation. Both animals present normal and homogeneous ventilation throughout both lungs. I & J: Normalized hp Xe-129 SB-CSI maps at 8 weeks. While the animal from the radioprotector group (I) shows a uniform distribution of the hp Xe-129 dissolved in the lung tissue (small elevation in the left lung upper lobe from pericardial fat-arrow), the animal from the control group (J) shows an elevation of the hp Xe-129 dissolved in the lung tissue due to a ventilation-perfusion mismatch caused by the radiation (arrow). Note the good correlation between the elevated area in J and the perfusion defect in F.

In order to further study the efficacy of this method in a very controlled manner and create ventilation-perfusion maps (V/Q maps), identical to the ones created using nuclear medicine techniques, six rabbits had an angio-catheter with a balloon placed in their left pulmonary artery under fluoroscopic guidance (FIG. 3A). This balloon permitted the full occlusion of the left pulmonary artery (FIG. 3B). Each animal was scanned during two separate inhalations of hp Xe-129, one prior to occlusion and one post occlusion. This way, we were able to further demonstrate in-vivo the sensitivity of our new SB-CSI technique to the amount of pulmonary blood (FIGS. 3E, 3F), while ventilation remained constant (FIGS. 3G, 3H). We then created a ventilation-perfusion map (FIGS. 3I, 3J), with the images 3E-3H, all acquired in a single breath-hold of hp Xe-129, using SB-CSI.

More specifically, FIGS. 3A-3J shows a rabbit model of PE. All images are from the same animal. A: X-ray obtained during balloon (black arrow) occlusion of the left pulmonary artery. B: Contrast-enhanced perfusion MRI shows the right lung (RL) with normal perfusion while the left lung is not perfused (white arrow). C: MR spectrum from a single voxel of a SB-CSI acquisition shows the combined peak for hp Xe-129 dissolved in tissue and blood (center) and the gas peak (right); Gaussian curve fits are in red. D: The 32×32 matrix overlying an hp Xe-129 ventilation image shows the placement of voxels for the SB-CSI acquisition. Images E, G and I were obtained during occlusion; Images F, H and J were obtained before occlusion. E and I show the left lung with a reduced amount of hp Xe-129 dissolved in the blood and tissue, while F and J show both lungs with identical signal. G and H show identical, unchanged ventilation maps for both lungs during and before occlusion.

In summary, we developed a system and method to detect and assess in a single breath-hold the physiologic, morphologic and structural changes in multiple organs. The novel method described here as Single breath-hold 2D, 3D or 4D Chemical Shift Imaging (SB-CSI) with hyperpolarized Xe-129 (hp Xe-129), will likely show very high sensitivity for the detection of a large number of pathologies, with minimal or no side-effects, in a very comfortable and fast way. When compared to established methods including, but not limited to, nuclear medicine techniques, in particular ventilation-perfusion scans (V/Q), single photon emission computed tomography (SPECT), positron emission tomography (PET) or computed tomography (CT) with radio-contrast, the SB-CSI with hp Xe-129 has several advantageous points: (i) no use of ionizing radiation; (ii) no requirement for the inhalation and/or intra-venous (IV) injection of radioactive tracers that can produce serious side-effects, like damage of cellular DNA; (iii) higher image resolutions than the first three nuclear medicine techniques mentioned above; (iii) data acquisition acquired in a single breath-hold instead of several minutes as with all the nuclear medicine techniques mentioned above.

This system, computer program product and method may provide improved sensitivity for the detection and monitoring or diseases and conditions including, but not limited to, tissue inflammation, tumors (malign and benign), other forms of cancer that don't comprise masses, pulmonary embolism and tissue edema. Since hp Xe-129 dissolves into tissue and binds to red blood cells, which carry it throughout the body, it can be used not only to assess changes in the lung but in any other organ. Also since it does not involve the use of ionizing radiation and does not have severe side-effects, it can be used multiple times daily, or repeated as desired to determine and monitor the progression of disease or to evaluate the efficacy of therapy.

An aspect of various embodiments may comprise a system, computer program product and method to detect and assess in a single breath-hold the physiologic, morphologic and structural changes in multiple organs. Current technologies using hyperpolarized gases, magnetic resonance imaging (MRI), magnetic resonance spectroscopy (MRS) or nuclear medicine techniques, including but not limited to, ventilation-perfusion (V/Q) scans, SPECT, PET and CT with radioactive contrast, can only obtain part of the physiologic or morphologic information that we can obtain with the novel SB-CSI system and method in a single breath-hold. An embodiment does not use ionizing radiation unlike all the other methods that currently provide identical physiologic and/or morphologic information. Furthermore, ionizing radiation carries severe side-effects and is not recommended for use on a short-time basis (less than 6 months), nor is it recommended for use in children or pregnant women unless medically necessary. The system and method presented here can be repeated as desired and used multiple times daily, if necessary, to determine and monitor the progression of disease or to evaluate the efficacy of therapy. When compared with other current methods that use hp Xe-129, all the other methods are done in multiple breath-holds and all methods except CSI can only attain pulmonary information. Our novel system, computer program product and method, can obtain physiologic, morphologic and structural information in any organ, in a simple, functional, and comfortable way for the subject.

This system, computer program product and method may provide improved sensitivity for the detection and monitoring diseases or conditions including, but not limited to, tissue inflammation, tumors (malign and benign), other forms of cancer that don't comprise masses, pulmonary embolism, and tissue edema.

When compared to established methods like, but not limited to, nuclear medicine techniques, in particular ventilation/perfusion scans (V/Q), single photon emission computed tomography (SPECT), positron emission tomography (PET) or computed tomography (CT) with radio-contrast, the SB-CSI with hp Xe-129 has several advantageous points: (i) no use of ionizing radiation; (ii) no requirement for the inhalation and/or intra-venous (IV) injection of radioactive tracers that can produce serious side-effects, like damage of cellular DNA; (iii) higher image resolutions than the first three nuclear medicine techniques mentioned above; (iii) data acquisition acquired in a single breath-hold instead of several minutes as with all the nuclear medicine techniques mentioned above. Also since it does not involve the use of ionizing radiation and does not have severe side-effects, it can be used multiple times daily, or repeated as desired to determine and monitor the progression of disease or to evaluate the efficacy of therapy.

Since the available alternatives, in particular the ventilation/perfusion scans (V/Q), single photon emission computed tomography (SPECT), positron emission tomography (PET) or computed tomography (CT) use ionizing radiation and the first three produce images with low resolution, this new method and system can in the long term replace completely at least the V/Q scans, and complement some of the other techniques that also use very specific tracers capable of target specific cell mechanisms. In a conservative model where this system and technique could replace the V/Q scans only, this alone would be a multi-billion dollar market, since most of the hospitals in the United States and around the world have nuclear medicine V/Q scan equipment and each scanner can cost more than a million dollars, not to mention the costs associated with the radioactive tracers.

For the other side, since most hospitals in the US and in developed countries have at least one MRI scanner, this system and method could be easily implemented, and the costs associated with extra hardware and hp Xe-129 could be less than the current costs associated with nuclear medicine V/Q scans, which could be a very attractive tool for an increasing global health system that pursues better diagnostic tools associated with lower costs.

Another embodiment will now be disclosed. Previous implementations of 2D-CSI of the lung with hyperpolarized xenon-129 (Xe-129) have been demonstrated (32). Here, we report the preliminary evaluation of this optimized technique using a rabbit model of lung fibrosis and another of emphysema. We also report the acquisition of multiple contiguous slices with a 3D-CSI version that covers the entire lung in a breath-hold.

From the CSI data, we directly calculate images reflecting the amount of Xe-129 in the airspaces, and dissolved in the lung tissue, Red Blood Cells (RBC), and other compartments, thus obtaining detailed spatial information regarding how Xe-129 is distributed in those different compartments and providing regional information about lung physiology.

Three New Zealand White rabbits underwent hyperpolarized Xe-129 2D or 3D-CSI one four different days: one before and 2, 4 and 7 weeks after the endotracheal instillation of bleomycin which induces lung fibrosis, or after the endotracheal instillation of porcine elastase which initially induces lung inflammation but later induces emphysema, and one control animal with no intervention. All scans were done in a 1.5 Tesla clinical system (Avanto, Siemens Medical Solutions) using a transmitter/receiver birdcage RF coil (IGC Medical Advances, Milwaukee, Wis.) tuned to the Xe-129 frequency. Isotopically enriched (˜87%) Xe-129 was polarized to ˜35% using a commercial prototype polarizer (Xemed LLC, NH). For each 2D or 3D acquisition, a single 40-cc volume was administered to the animal, and respiration was suspended for the acquisition (˜6-18s). For the 2D-projection CSI acquisition, a matrix of 26×26 voxels, interpolated to 32×32 voxels, was positioned over the lungs, with a FOV of 90×90 mm², corresponding to an in-plane resolution of 2.8×2.8 mm² and TR/TE=27/2.3 ms. For the 3D-CSI acquisition, a matrix of 16×16×8 voxels, interpolated to 32×32×8 was used, with a FOV of 110×110×110 mm³. For each excitation an RF pulse with duration 1280 μs and bandwidth 3125 Hz was applied at the frequency of the dissolved-phase Xe-129 (˜200 ppm from Xe-129 gas).

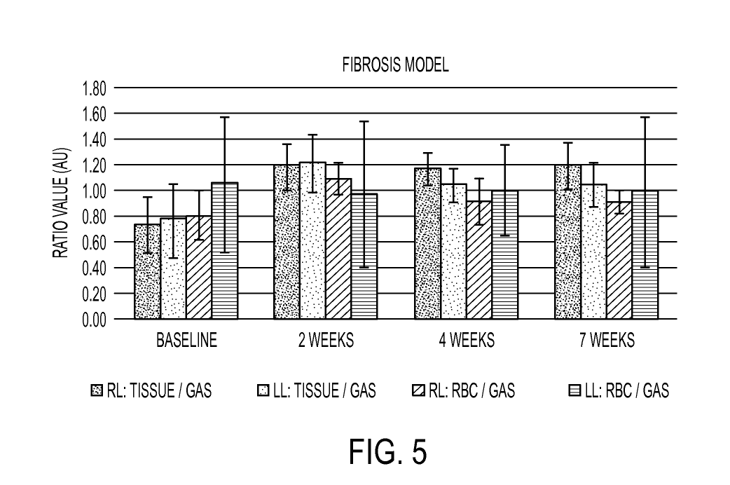

Quantification of the maps for the animal in the fibrosis model group show an increase in the tissue/gas and RBC/gas ratios following the instillation of bleomycin, but only the tissue/gas remained elevated, as expected in fibrosis (FIG. 5).

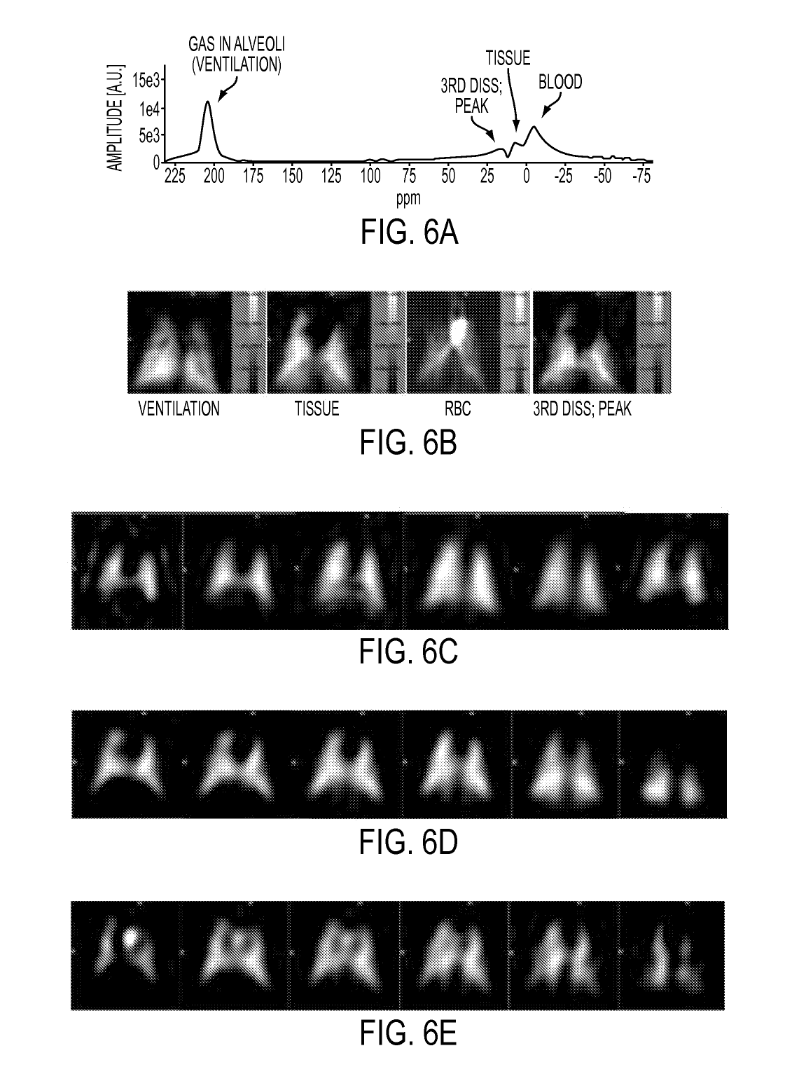

High-resolution 2D-CSI maps of the animal in the lung fibrosis group showed the presence of a third dissolved-phase peak at about 185 ppm from the alveolar gas peak, and adjacent to the dissolved-phase tissue peak (FIG. 6A, which shows a full spectrum, from a single voxel, obtained during a 2D-CSI acquisition of the rabbit in the fibrosis model group, at 7 weeks post). This Xe-129 dissolved-phase peak is not well resolved in normal rabbits and it was not seen in our control animal. 2D-CSI (FIG. 6B, which shows 2D-CSI maps for each of the peaks indicated in FIG. 6A) and 3D-CSI (FIGS. 6C, 6D, 6E, showing the gas-peak (ventilation) images, the tissue peak images, and the images from the RBC peak, respectively) map production and post-processing was performed using the 3DiCSI (QiZhao, Columbia University, NY) and MATLAB (MathWorks, Natick, Mass.) software packages. The free-induction decay (FID) corresponding to each voxel was filtered with a Gaussian, zero filled to 2048 points and Fourier transformed. Subsequently, each separate peak in the spectrum was quantified using a Principal Component Analysis (PCA) method (33)

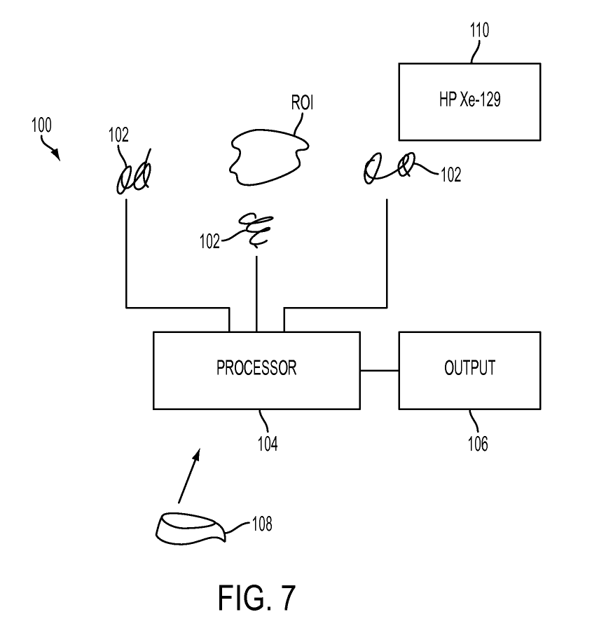

FIG. 7 shows an example of hardware on which the preferred embodiment or another embodiment can be implemented. The system 100 images a region of interest ROI using multiple coils 102. A processor 104 receives raw image data from the coils and processes them as disclosed herein to produce the images and to perform further processing. The images and the results of further processing are output to an output 106, which can include one or more of a display, a printer, persistent storage, and a communication device for transmitting the images and results of further processing to a remote location. Software for implementing the preferred embodiment or another embodiment can be supplied on any suitable machine-readable medium 108. Any appropriate source 110 of hyperpolarized Xe-129 can be used. It is contemplated that the image reconstruction will be performed in real time or near real time, although as an alternative, the raw image data could be taken and stored for later processing.

While preferred embodiments have been set forth above, those skilled in the art who have reviewed the present disclosure will readily appreciate that other embodiments can be realized within the scope of the invention. For example, numerical values are illustrative rather than limiting. Also, the invention is applicable to a wide variety of uses, including any suitable location in a human or non-human animal body or nonbiological uses. Therefore, the present invention should be construed as limited only by the appended claims.

We claim:

1. A method for assessing structural changes in a volume of interest, the method comprising:
(a) polarizing Xe-129 gas to form hyperpolarized Xe-129 gas;
(b) introducing the hyperpolarized Xe-129 gas into the volume of interest;
(c) acquiring multiple spatially oriented spectra from the volume of interest, using a magnetic resonance pulse sequence capable of producing said multiple spatially oriented spectra within a predetermined time;
(d) post-processing the multiple spatially oriented spectra in a computing device to obtain magnetic resonance spectra and chemical shift maps; and
(e) evaluating the magnetic resonance spectra, the chemical shift maps, or the magnetic resonance spectra and the chemical shift maps to assess the structural changes in the volume of interest.

2. The method of claim 1, wherein the volume of interest is in a lung of a patient.

3. The method of claim 2, wherein the predetermined time is less than a breath hold of the patient.

4. The method of claim 1, wherein the predetermined time is less than 15 seconds.

5. The method of claim 1, wherein the magnetic resonance pulse sequence has a repetition time which is minimized in accordance with the predetermined time.

6. The method of claim 1, wherein the magnetic resonance pulse sequence has crushing gradients whose times and amplitudes are selected in accordance with the predetermined time.

7. The method of claim 1, wherein the magnetic resonance pulse sequence has an echo time of less than 2.1 milliseconds.

8. The method of claim 1, wherein the magnetic resonance pulse sequence has an RF flip angle of less than 30 degrees per voxel.

9. A system for assessing structural changes in a volume of interest, the system comprising:
a source of hyperpolarized Xe-129 gas;
a processing device; and
a plurality of magnetic resonance coils, under control of the processing device, for acquiring multiple spatially oriented spectra from the volume of interest, using a magnetic resonance pulse sequence capable of producing said multiple spatially oriented spectra within a predetermined time;
the processing device being configured for post-processing the multiple spatially oriented spectra in a computing device to obtain magnetic resonance spectra and chemical shift maps and evaluating the magnetic resonance spectra, the chemical shift maps, or the magnetic resonance spectra and the chemical shift maps to assess the structural changes in the volume of interest.

10. The system of claim 9, wherein processing device is configured for use in situations in which the volume of interest is in a lung of a patient.

11. The system of claim, wherein the processing is configured such that the predetermined time is less than a breath hold of the patient.

12. The system of claim 9, wherein the processing device is configured such that the predetermined time is less than 15 seconds.

13. The system of claim 9, wherein the processing device is configured such that the magnetic resonance pulse sequence has a repetition time which is minimized in accordance with the predetermined time.

14. The system of claim 9, wherein the processing device is configured such that the magnetic resonance pulse sequence has crushing gradients whose times and amplitudes are selected in accordance with the predetermined time.

15. The system of claim 9, wherein the processing device is configured such that the magnetic resonance pulse sequence has an echo time of less than 2.1 milliseconds.

16. The system of claim 9, wherein the processing device is configured such that the magnetic resonance pulse sequence has an RF flip angle of less than 30 degrees per voxel.

17. An article of manufacture for assessing structural changes in a volume of interest, the article of manufacture comprising:
a computer-readable storage medium; and
code stored on the computer-readable storage medium, the code, when executed on a processing device, controlling the processing device for:
acquiring multiple spatially oriented spectra from the volume of interest after hyperpolarized Xe-129 gas has been introduced into the volume of interest, using a magnetic resonance pulse sequence capable of producing said multiple spatially oriented spectra within a predetermined time;
post-processing the multiple spatially oriented spectra in a computing device to obtain magnetic resonance spectra and chemical shift maps; and
evaluating the magnetic resonance spectra, the chemical shift maps, or the magnetic resonance spectra and the chemical shift maps to assess the structural changes in the volume of interest.

18. The article of manufacture of claim 17, wherein the code is written for use in situations in which the volume of interest is in a lung of a patient.

19. The article of manufacture of claim 18, wherein the code is written such that the predetermined time is less than a breath hold of the patient.

20. The article of manufacture of claim 17, wherein the code is written such that the predetermined time is less than 15 seconds.

21. The article of manufacture of claim 17, wherein the code is written such that the magnetic resonance pulse sequence has a repetition time which is minimized in accordance with the predetermined time.

22. The article of manufacture of claim 17, wherein the code is written such that the magnetic resonance pulse sequence has crushing gradients whose times and amplitudes are selected in accordance with the predetermined time.

23. The article of manufacture of claim 17, wherein the code is written such that the magnetic resonance pulse sequence has an echo time of less than 2.1 milliseconds.

24. The article of manufacture of claim 17, wherein the code is written such that the magnetic resonance pulse sequence has an RF flip angle of less than 30 degrees per voxel.
